(19) United States
(12) Patent Application Publication
(10) Pub. No.: US 2025/0017767 A1
(43) Pub. Date: Jan. 16, 2025

(54) Heated Body Blanket Device

(71) Applicant: Sarah Birdsong, Batesville, MS (US)

(72) Inventor: Sarah Birdsong, Batesville, MS (US)

(21) Appl. No.: 18/220,031

(22) Filed: Jul. 10, 2023

(51) Int. Cl. A61F 7/00; H05B 3/34

(52) U.S. Cl. CPC A61F 7/0097; A61F 7/007; H05B 3/342; A61F 2007/0078; A61F 2007/0086

(57) ABSTRACT

A heated body blanket device includes a blanket that has a cut extending through the blanket so a user's body can be extended through the cut. The cut defines a first portion of the blanket and a second portion of the blanket which can be wrapped around the user's torso or positioned over the user's legs. A first heating element is integrated into the first portion of the blanket to warm the user's torso. A second heating element is integrated into the second portion of the blanket to warm the user's legs. A control unit is attached to the blanket and the control unit independently actuates or de-actuates the first heating element and the second heating element. In this way the control unit facilitates the user to either warm their torso or their legs.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to blanket devices and more particularly pertains to a new blanket device for facilitating a user to simultaneously or independently warm a user's torso and the user's legs. The device includes a blanket with a lateral cut that a user can extend their body through to wrap the blanket around their torso and cover their legs with the blanket. The device includes a pair of heating elements that are each integrated into a respective portion of the blanket which are independently operated by a control unit for warming the user's torso or legs simultaneously or independently.

### (2) Description of Related Art Including Information Disclosed Under 37 CFR 1.97 and 1.98

The prior art relates to blanket devices including a variety of bodily warming devices that includes blankets with heating elements, a poncho with heating elements and a hooded garment with heating elements. In no instance does the prior art disclose a blanket with a cut through which a user's body can be extended for covering the user's torso and the user's legs and pair of heating elements that are independently operated.

### BRIEF SUMMARY OF THE INVENTION

An embodiment of the disclosure meets the needs presented above by generally comprising a blanket that has a cut extending through the blanket so a user's body can be extended through the cut. The cut defines a first portion of the blanket and a second portion of the blanket which can be wrapped around the user's torso or positioned over the user's legs. A first heating element is integrated into the first portion of the blanket to warm the user's torso. A second heating element is integrated into the second portion of the blanket to warm the user's legs. A control unit is attached to the blanket and the control unit independently actuates or de-actuates the first heating element and the second heating element. In this way the control unit facilitates the user to either warm their torso or their legs.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front view of a heated body blanket device according to an embodiment of the disclosure.

FIG. 2 is a back phantom view of an embodiment of the disclosure.

FIG. 3 is a back perspective view of an embodiment of the disclosure.

FIG. 4 is a cross sectional view taken along line 4-4 of FIG. 2 of an embodiment of the disclosure.

FIG. 5 is a back perspective in-use view of an embodiment of the disclosure.

FIG. 6 is a front perspective in-use view of an embodiment of the disclosure.

FIG. 7 is a perspective in-use view of an embodiment of the disclosure.

FIG. 8 is a schematic view of an embodiment of the disclosure.

### DETAILED DESCRIPTION OF THE INVENTION

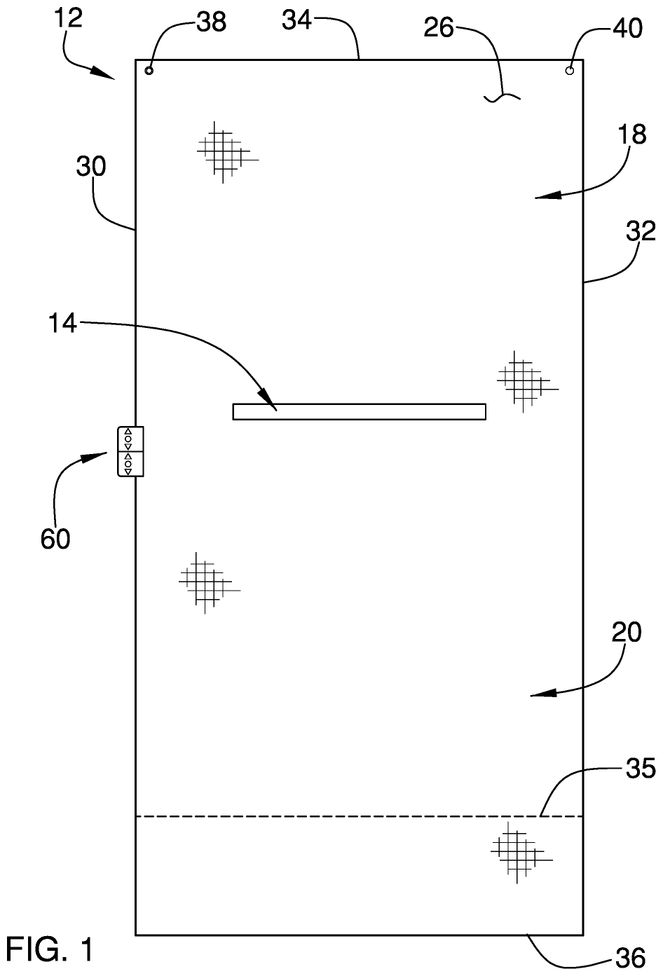

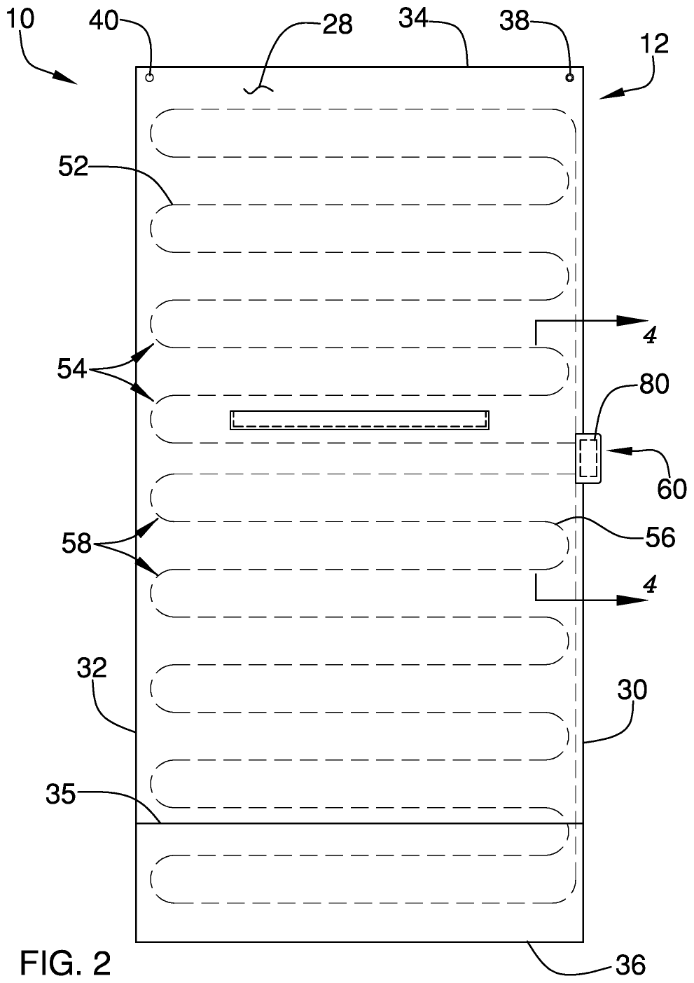

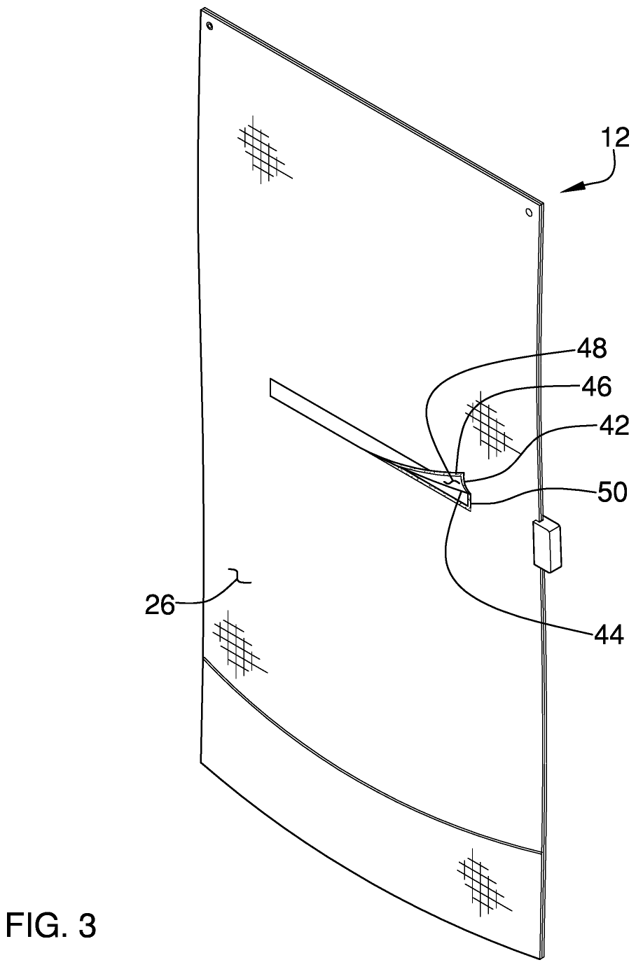

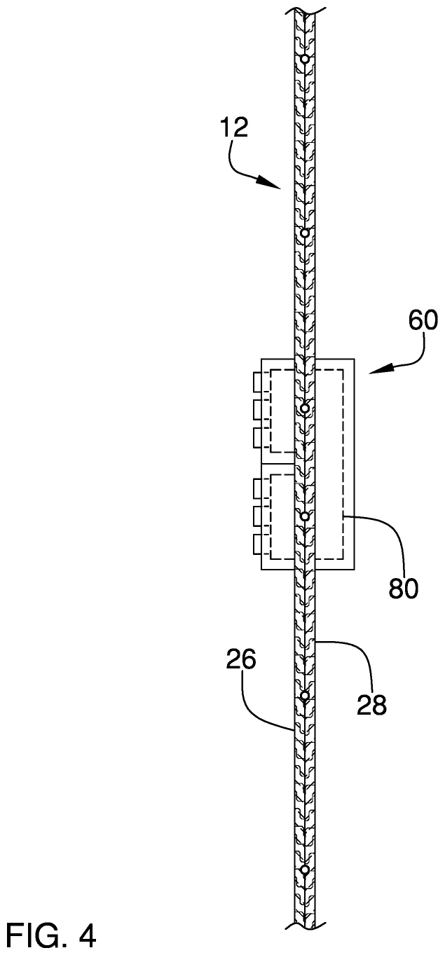

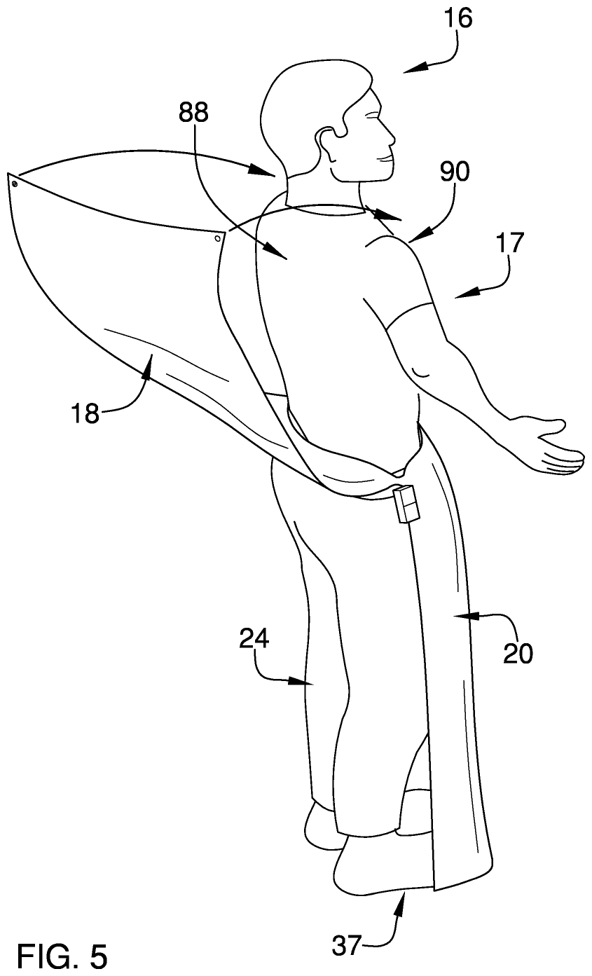

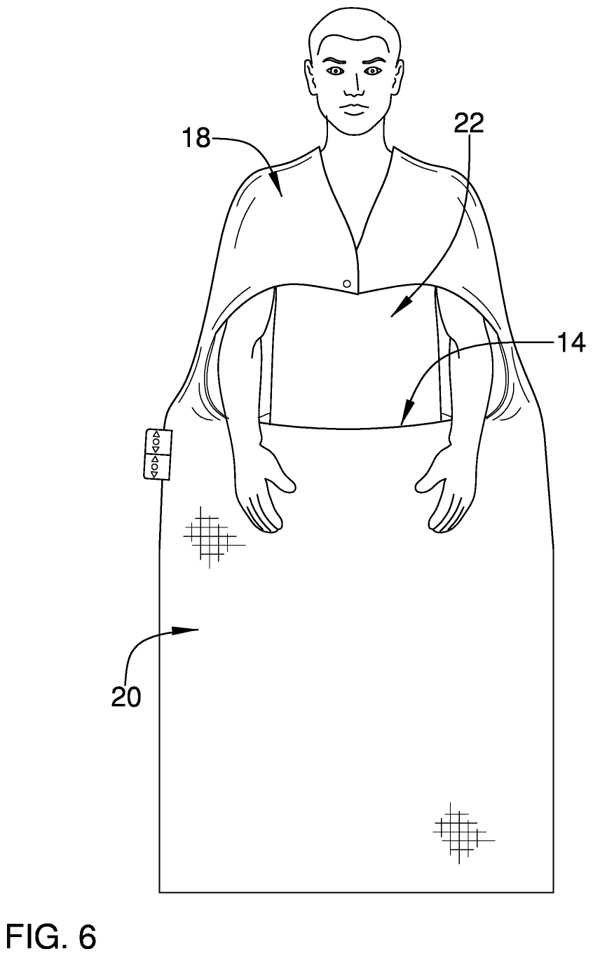

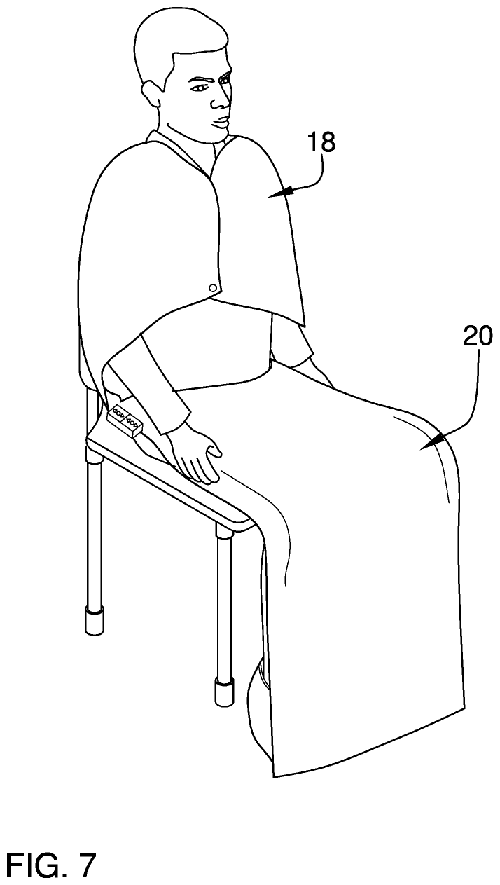

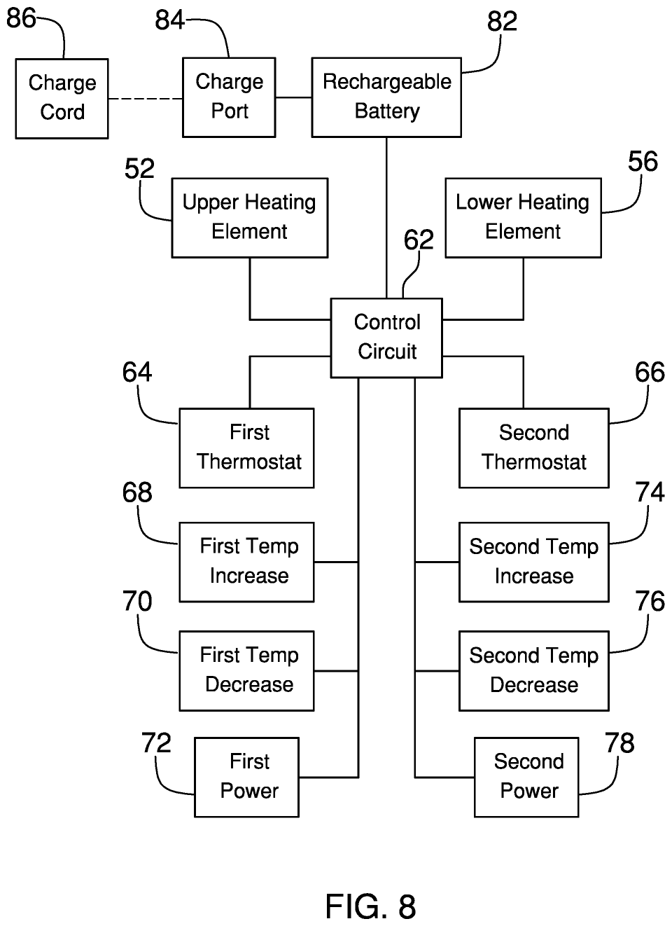

With reference now to the drawings, and in particular to FIGS. 1 through 8 thereof, a new blanket device embodying the principles and concepts of an embodiment of the disclosure and generally designated by the reference numeral 10 will be described.

As best illustrated in FIGS. 1 through 8, the heated body blanket device 10 generally comprises a blanket 12 that has a cut 14 extending through the blanket 12 such that a user 16 can extend their body 17 through the cut 14. The cut 14 is centrally located on the blanket 12 such that the cut 14 defines a first portion 18 of the blanket 12 and a second portion 20 of the blanket 12. Furthermore, the first portion 18 can be wrapped around the user's torso 22 and the second portion 20 can be positioned over the user's legs 24. The cut 14 extends through a top surface 26 and a bottom surface 28 of the blanket 12 and the cut 14 extends along an axis extending between a first lateral edge 30 and a second lateral edge 32 of the blanket 12. Additionally, the cut 14 is centrally positioned between a top edge 34 and a bottom edge 36 of the blanket 12. The blanket 12 may be comprised of a thermally insulating textile, including but not being limited to, cotton or wool. A pocket 35 is attached to the bottom surface 28 of the blanket 12 and extends along the bottom edge 36 of the blanket 12 for receiving the user's feet 37.

A first coupler 38 is attached to the blanket 12 and the first coupler 38 is positioned on the first portion 18 of the blanket 12. Additionally, the first coupler 38 is aligned with an intersection between the top edge 34 and the first lateral edge 30 of the blanket 12. A second coupler 40 is attached to the blanket 12 and the second coupler 40 is positioned on the first portion 18 of the blanket 12. The first coupler 38 is releasably matable to the second coupler 40 to retain the first portion 18 of the blanket 12 around the user's torso 22. Additionally, the second coupler 40 is aligned with an intersection between the top edge 34 and the second lateral edge 32 of the blanket 12. Each of the first coupler 38 and the second coupler 40 may comprise snaps, buttons or other types of complementary mechanical couplers.

A flap 42 is coupled to the blanket 12 and the flap 42 is positionable over the cut 14 for closing the cut 14. The flap 42 has a first edge 44 that is coupled to the bottom surface 28 of the blanket 12 and the first edge 44 is aligned with and extends along the cut 14. A first mating member 46 is coupled to a lower surface 48 of the flap 42 and the first mating member 46 extends partially around a perimeter of the flap 42. A second mating member 50 is coupled to the bottom surface 28 of the blanket 12 and the second mating member 50 extends partially around the cut 14. The first mating member 46 is releasably matable to the second mating member 50 for retaining the flap 42 over the cut 14. Each of the first mating member 46 and the second mating member 50 may comprise a hook and loop fastener or other type of multiple use, releasable fastener.

A first heating element 52 is integrated into the first portion 18 of the blanket 12. The first heating element 52 heats the first portion 18 when the first heating element 52 is turned on. In this way the first heating element 52 can warm the user's torso 22. The first heating element 52 is positioned between the top surface 26 and the bottom surface 28 of the blanket 12. The first heating element 52 comprises a plurality of coils 54 extending between the first lateral edge 30 and the second lateral edge 32 of the blanket 12. Additionally, the coils 54 are spaced apart from each other and are evenly distributed between the top edge 34 and the cut 14. The first heating element 52 may comprise an electronic heating element or the like with an operational temperature ranging between approximately 80.0 degrees Fahrenheit and 100.0 degrees Fahrenheit.

A second heating element 56 is integrated into the second portion 20 of the blanket 12. The second heating element 56 heats the second portion 20 when the second heating element 56 is turned on. In this way the second heating element 56 can warm the user's legs 24. The second heating element 56 is positioned between the top surface 26 and the bottom surface 28 of the blanket 12 and the second heating element 56 comprises a plurality of coils 58 extending between the first lateral edge 30 and the second lateral edge 32 of the blanket 12. The coils 58 associated with the second heating element 56 are spaced apart from each other and are evenly distributed between the bottom edge 36 and the cut 14. The second heating element 56 may comprise an electronic heating element or the like with an operational temperature ranging between approximately 80.0 degrees Fahrenheit and 100.0 degrees Fahrenheit.

A control unit 60 is attached to the blanket 12 such that the control unit 60 is accessible to the user 16. The control unit 60 is in communication with each of the first heating element 52 and the second heating element 56. Furthermore, the control unit 60 independently actuates or de-actuates the first heating element 52 and the second heating element 56 to facilitate the user 16 to either warm their torso or their legs. The control unit 60 comprises a control circuit 62 that is integrated into the control unit 60 and the control circuit 62 is electrically coupled to the first heating element 52 and the control circuit 62 is electrically coupled to the second heating element 56.

The control unit 60 includes a first thermostat 64 that is integrated into the first portion 18 of the blanket 12 such that the first thermostat 64 is in thermal communication with the first portion 18 of the blanket 12. Additionally, the first thermostat 64 is electrically coupled to the control circuit 62. The control unit 60 includes a second thermostat 66 that is integrated into the second portion 20 of the blanket 12 such that the second thermostat 66 is in thermal communication with the second portion 20 of the blanket 12. Additionally, the second thermostat 66 is electrically coupled to the control circuit 62. Each of the first thermostat 64 and the second thermostat 66 may comprise an electronic thermostat or the like.

The control unit 60 includes a first temperature increase button 68 that is movably integrated into the control unit 60 and the first temperature increase button 68 is electrically coupled to the control circuit 62. The first temperature increase button 68 increases an operational temperature of the first heating element 52 toward a maximum temperature when the first temperature increase button 68 is depressed. The control unit 60 includes a first temperature decrease button 70 that is movably integrated into the control unit 60. The first temperature decrease button 70 is electrically coupled to the control circuit 62 and the first temperature decrease button 70 decreases an operational temperature of the first heating element 52 toward a minimum temperature when the first temperature decrease button 70 is depressed. The control unit 60 includes a first power button 72 that is movably integrated into the control unit 60. The first power button 72 is electrically coupled to the control circuit 62 and the first power button 72 turns the first heating element 52 on and off.

The control unit 60 includes a second temperature increase button 74 that is movably integrated into the control unit 60 and the second temperature increase button 74 is electrically coupled to the control circuit 62. The second temperature increase button 74 increases an operational temperature of the second heating element 56 toward a maximum temperature when the second temperature increase button 74 is depressed. The control unit 60 includes a second temperature decrease button 76 that is movably integrated into the control unit 60 and the second temperature decrease button 76 is electrically coupled to the control circuit 62. The second temperature decrease button 76 decreases an operational temperature of the second heating element 56 toward a minimum temperature when the second temperature decrease button 76 is depressed. The control unit 60 includes a second power button 78 that is movably integrated into the control unit 60. The second power button 78 is electrically coupled to the control circuit 62 and the second power button 78 turns the second heating element 56 on and off.

The control circuit 62 alternatively actuates and de-actuates the first heating element 52 according to the temperature sensed by the first thermostat 64 for maintaining the temperature of the first portion 18 of the blanket 12 at a preferred temperature. The control circuit 62 alternatively actuates and de-actuates the second heating element 56 according to the temperature sensed by the second thermostat 66 for maintaining the temperature of the second portion 20 of the blanket 12 at a preferred temperature. A power supply 80 is integrated into the control unit 60 and the power supply 80 is electrically coupled to the control circuit 62. The power supply 80 comprises a rechargeable battery 82 that is integrated into the control unit 60 and the rechargeable battery 82 is electrically coupled to the control circuit 62. The power supply 80 includes a charge port 84 that is recessed into the control unit 60 thereby facilitating the charge port 84 to receive a charge cord 86. The charge port 84 is electrically coupled to the rechargeable battery 82 for charging the rechargeable battery 82.

In use, the user 16 extends their body 17 through the cut 14 and wraps the first portion 18 of the blanket 12 upwardly over their back 88 and downwardly over their shoulders 90 and subsequently attaches the first coupler 38 to the second coupler 40. In this way the first portion 18 covers the user's torso 22 and the second portion 20 covers the user's legs 24. The control unit 60 is manipulated to actuate either the first heating element 52 or the second heating element 56 independently or simultaneously, depending on the user's 16 preference. In this way the user's torso 22, especially their back 88, can be warmed and the user's legs 24 can be warmed either independently or simultaneously. Additionally, the temperature of the first heating element 52 and the second heating element 56 can be adjusted to suit the user's 16 preference. The flap 42 can be closed over the cut 14 to facilitate the user 16 to cover their body 17 with the blanket 12 without extending their body 17 through the cut 14.

With respect to the above description then, it is to be realized that the optimum dimensional relationships for the parts of an embodiment enabled by the disclosure, to include variations in size, materials, shape, form, function and manner of operation, device and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A heated body blanket device being wearable around a user's torso and over the user's legs, said device comprising:
a blanket having a cut extending through said blanket wherein said cut is configured to have a user's body extended through said cut, said cut being centrally located on said blanket such that said cut defines a first portion of said blanket and a second portion of said blanket wherein said first portion is configured to be wrapped around the user's torso and wherein said second portion is configured to be positioned over the user's legs;
a first coupler being attached to said blanket, said first coupler being positioned on said first portion of said blanket;
a second coupler being attached to said blanket, said second coupler being positioned on said first portion of said blanket, said first coupler being releasably matable to said second coupler wherein said first coupler and said second coupler are configured to retain said first portion of said blanket around the user's torso;
a flap being coupled to said blanket, said flap being positionable over said cut for closing said cut;
a first heating element being integrated into said first portion of said blanket, said first heating element heating said first portion when said first heating element is turned on wherein said first heating element is configured to warm the user's torso;
a second heating element being integrated into said second portion of said blanket, said second heating element heating said second portion when said second heating element is turned on wherein said second heating element is configured to warm the user's legs; and
a control unit, said control unit being attached to said blanket wherein said control unit is configured to be accessible to the user, said control unit being in communication with each of said first heating element and said second heating element, said control unit independently actuating or de-actuating said first heating element and said second heating element wherein said control unit is configured to facilitate the user to either warm their torso or their legs.

2. The device according to claim 1, wherein:
said cut extends through a top surface and a bottom surface of said blanket, said cut extending along an axis extending between a first lateral edge and a second lateral edge of said blanket, said cut being centrally positioned between a top edge and a bottom edge of said blanket;
said first coupler is aligned with an intersection between said top edge and said first lateral edge of said blanket; and
said second coupler is aligned with an intersection between said top edge and said second lateral edge of said blanket.

3. The device according to claim 1, wherein:
said flap has a first edge being coupled to a bottom surface of said blanket, said first edge being aligned with and extending along said cut;
said device includes a first mating member being coupled to a lower surface of said flap, said first mating member extending partially around a perimeter of said flap; and
said device includes a second mating member being coupled to said bottom surface of said blanket, said second mating member partially extending around said cut, said first mating member being releasably matable to said second mating member for retaining said flap over said cut.

4. The device according to claim 2, wherein said first heating element is positioned between said top surface and said bottom surface of said blanket, said first heating element comprising a plurality of coils extending between said first lateral edge and said second lateral edge of said blanket, said coils being spaced apart from each other and being evenly distributed between said top edge and said cut.

5. The device according to claim 2, wherein said second heating element is positioned between said top surface and said bottom surface of said blanket, said second heating element comprising a plurality of coils extending between said first lateral edge and said second lateral edge of said blanket, said coils associated with said second heating element being spaced apart from each other and being evenly distributed between said bottom edge and said cut.

6. The device according to claim 1, wherein said control unit comprises:
a control circuit being integrated into said control unit, said control circuit being electrically coupled to said first heating element, said control circuit being electrically coupled to said second heating element;
a first thermostat being integrated into said first portion of said blanket such that said first thermostat is in thermal communication with said first portion of said blanket, said first thermostat being electrically coupled to said control circuit; and
a second thermostat being integrated into said second portion of said blanket such that said second thermostat is in thermal communication with said second portion of said blanket, said second thermostat being electrically coupled to said control circuit.

7. The device according to claim 6, wherein said control unit includes:
a first temperature increase button being movably integrated into said control unit, said first temperature increase button being electrically coupled to said control circuit, said first temperature increase button increasing an operational temperature of said first heating element toward a maximum temperature when said first temperature increase button is depressed;
a first temperature decrease button being movably integrated into said control unit, said first temperature decrease button being electrically coupled to said control circuit, said first temperature decrease button decreasing an operational temperature of said first heating element toward a minimum temperature when said first temperature decrease button is depressed; and
a first power button being movably integrated into said control unit, said first power button being electrically coupled to said control circuit, said first power button turning said first heating element on and off.

8. The device according to claim 7, wherein said control unit includes:
a second temperature increase button being movably integrated into said control unit, said second temperature increase button being electrically coupled to said control circuit, said second temperature increase button increasing an operational temperature of said second heating element toward a maximum temperature when said second temperature increase button is depressed;
a second temperature decrease button being movably integrated into said control unit, said second temperature decrease button being electrically coupled to said control circuit, said second temperature decrease button decreasing an operational temperature of said second heating element toward a minimum temperature when said second temperature decrease button is depressed; and
a second power button being movably integrated into said control unit, said second power button being electrically coupled to said control circuit, said second power button turning said second heating element on and off.

9. The device according to claim 6, wherein:
said control circuit alternatively actuates and de-actuates said first heating element according to the temperature sensed by said first thermostat for maintaining the temperature of said first portion of said blanket at a preferred temperature; and
said control circuit alternatively actuates and de-actuates said second heating element according to the temperature sensed by said second thermostat for maintaining the temperature of said second portion of said blanket at a preferred temperature.

10. The device according to claim 6, further comprising a power supply being integrated into said control unit, said power supply being electrically coupled to said control circuit, said power supply comprising:
a rechargeable battery being integrated into said control unit, said rechargeable battery being electrically coupled to said control circuit; and
a charge port being recessed into said control unit thereby facilitating said charge port to receive a charge cord, said charge port being electrically coupled to said rechargeable battery for charging said rechargeable battery.

11. A heated body blanket device being wearable around a user's torso and over the user's legs, said device comprising:
a blanket having a cut extending through said blanket wherein said cut is configured to have a user's body extended through said cut, said cut being centrally located on said blanket such that said cut defines a first portion of said blanket and a second portion of said blanket wherein said first portion is configured to be wrapped around the user's torso and wherein said second portion is configured to be positioned over the user's legs, said cut extending through a top surface and a bottom surface of said blanket, said cut extending along an axis extending between a first lateral edge and a second lateral edge of said blanket, said cut being centrally positioned between a top edge and a bottom edge of said blanket;
a first coupler being attached to said blanket, said first coupler being positioned on said first portion of said blanket, said first coupler being aligned with an intersection between said top edge and said first lateral edge of said blanket;
a second coupler being attached to said blanket, said second coupler being positioned on said first portion of said blanket, said first coupler being releasably matable to said second coupler wherein said first coupler and said second coupler are configured to retain said first portion of said blanket around the user's torso, said second coupler being aligned with an intersection between said top edge and said second lateral edge of said blanket;
a flap being coupled to said blanket, said flap being positionable over said cut for closing said cut, said flap having a first edge being coupled to said bottom surface of said blanket, said first edge being aligned with and extending along said cut;
a first mating member being coupled to a lower surface of said flap, said first mating member extending partially around a perimeter of said flap;
a second mating member being coupled to said bottom surface of said blanket, said second mating member partially extending around said cut, said first mating member being releasably matable to said second mating member for retaining said flap over said cut;
a first heating element being integrated into said first portion of said blanket, said first heating element heating said first portion when said first heating element is turned on wherein said first heating element is configured to warm the user's torso, said first heating element being positioned between said top surface and said bottom surface of said blanket, said first heating element comprising a plurality of coils extending between said first lateral edge and said second lateral edge of said blanket, said coils being spaced apart from each other and being evenly distributed between said top edge and said cut;
a second heating element being integrated into said second portion of said blanket, said second heating element heating said second portion when said second heating element is turned on wherein said second heating element is configured to warm the user's legs, said second heating element being positioned between said top surface and said bottom surface of said blanket, said second heating element comprising a plurality of coils extending between said first lateral edge and said second lateral edge of said blanket, said coils associated with said second heating element being spaced apart from each other and being evenly distributed between said bottom edge and said cut; and
a control unit, said control unit being attached to said blanket wherein said control unit is configured to be accessible to the user, said control unit being in communication with each of said first heating element and said second heating element, said control unit independently actuating or de-actuating said first heating element and said second heating element wherein said control unit is configured to facilitate the user to either warm their torso or their legs, said control unit comprising:
a control circuit being integrated into said control unit, said control circuit being electrically coupled to said first heating element, said control circuit being electrically coupled to said second heating element;
a first thermostat being integrated into said first portion of said blanket such that said first thermostat is in thermal communication with said first portion of said blanket, said first thermostat being electrically coupled to said control circuit;
a second thermostat being integrated into said second portion of said blanket such that said second thermostat is in thermal communication with said second portion of said blanket, said second thermostat being electrically coupled to said control circuit;
a first temperature increase button being movably integrated into said control unit, said first temperature increase button being electrically coupled to said control circuit, said first temperature increase button increasing an operational temperature of said first heating element toward a maximum temperature when said first temperature increase button is depressed;
a first temperature decrease button being movably integrated into said control unit, said first temperature decrease button being electrically coupled to said control circuit, said first temperature decrease button decreasing an operational temperature of said first heating element toward a minimum temperature when said first temperature decrease button is depressed;
a first power button being movably integrated into said control unit, said first power button being electrically coupled to said control circuit, said first power button turning said first heating element on and off;
a second temperature increase button being movably integrated into said control unit, said second temperature increase button being electrically coupled to said control circuit, said second temperature increase button increasing an operational temperature of said second heating element toward a maximum temperature when said second temperature increase button is depressed;
a second temperature decrease button being movably integrated into said control unit, said second temperature decrease button being electrically coupled to said control circuit, said second temperature decrease button decreasing an operational temperature of said second heating element toward a minimum temperature when said second temperature decrease button is depressed;
a second power button being movably integrated into said control unit, said second power button being electrically coupled to said control circuit, said second power button turning said second heating element on and off;
said control circuit alternatively actuating and de-actuating said first heating element according to the temperature sensed by said first thermostat for maintaining the temperature of said first portion of said blanket at a preferred temperature;
said control circuit alternatively actuating and de-actuating said second heating element according to the temperature sensed by said second thermostat for maintaining the temperature of said second portion of said blanket at a preferred temperature; and
a power supply being integrated into said control unit, said power supply being electrically coupled to said control circuit, said power supply comprising:
a rechargeable battery being integrated into said control unit, said rechargeable battery being electrically coupled to said control circuit; and
a charge port being recessed into said control unit thereby facilitating said charge port to receive a charge cord, said charge port being electrically coupled to said rechargeable battery for charging said rechargeable battery.
